(19) United States
(12) Patent Application Publication
(10) Pub. No.: US 2005/0032674 A1
(43) Pub. Date: Feb. 10, 2005

(54) SAIF, an anti-inflammatory factor, and methods of use thereof

(76) Inventors: Ciaran P. Kelly, West Newton, MA (US); Charalabos Pothoulakis, Waban, MA (US); Stavros Sougioultzis, Brookline, MA (US); Killimangalam R. Bhaskar, Lexington, MA (US)

Correspondence Address: CLARK & ELBING LLP, 101 FEDERAL STREET, BOSTON, MA, 02110, US

(21) Appl. No.: 10/885,380

(22) Filed: Jul. 6, 2004

Related U.S. Application Data
(60) Provisional application No. 60/485,279, filed on Jul. 3, 2003.

(51) Int. Cl. A61K 38/16; A61K 31/715; C07K 14/47

(52) U.S. Cl. 514/8; 514/54; 530/395; 536/123.12

(57) ABSTRACT

The invention features a novel soluble anti-inflammatory factor (SAIF), methods of SAIF production and purification, and methods of using SAIF for the treatment or prevention of an inflammatory disease or disorder.

### CROSS REFERENCE TO RELATED APPLICATIONS

This application claims priority from U.S. Provisional Application No. 60/485,279, filed on Jul. 3, 2003, which is incorporated by reference in its entirety.

### BACKGROUND TO THE INVENTION

The present invention relates to the treatment of inflammatory diseases and conditions, such as arthritis, asthma, inflammatory bowel disease, acute or chronic gastrointestinal injury, and inflammation caused by infectious agents (e.g., bacterial, viral, or parasitic agents) or their toxins, and the treatment of injury and inflammation at extraintestinal sites, e.g., skin and the musculoskeletal system.

Saccharomyces boulardii (Sb) is a non-pathogenic yeast used for many years as a probiotic agent to prevent or treat a variety of human gastrointestinal disorders, including antibiotic associated diarrhea and recurrent Clostridium difficile disease (Elmer et al. JAMA 275:870-876, 1996 and Sullivan et al. 20:313-319, 2000). A recent report also suggests that Sb may be useful in preventing clinical relapse in Crohn's disease (Guslandi et al., Dig. Dis. Sci. 45:1462-1464, 2000). Several studies indicate that Sb may exert its beneficial effects by multiple mechanisms. For example, the protective effects of Sb on Clostridium difficile-induced inflammatory diarrhea appear to involve proteolytic digestion of C. difficile toxin A and B molecules by a secreted protease (Pothoulakis et al., Gastroenterology 104 :1108-1115, 1993, and Castagliuolo et al., Infect. Immun. 67:302-307, 1999). Competition with pathogens for nutrients, inhibition of pathogen adhesion, strengthening of enterocyte tight junctions, neutralization of bacterial virulence factors, and enhancement of the mucosal immune response are also among the reported potential mechanisms of action (Czerucka et al., Microbes. Infect. 4:733-739, 2002).

Chemokines are a superfamily of closely related chemoattractant cytokines that specialize in mobilizing leukocytes to areas of immune challenge. These inducible pro-inflammatory peptides potently stimulate leukocyte migration along a chemotactic gradient. IL-8 belongs to the C-X-C chemokine family and activates neutrophils by virtue of an E-L-R (Glu-Leu-Arg) amino acid motif that lies immediately adjacent to its C-X-C site. IL-8 is produced by many cell types including activated monocytes/macrophages, other leukocytes, endothelial cells and epithelial cells. IL-8, and other C-X-C chemokines, play a major role in regulating acute intestinal inflammation and neutrophil infiltration in C. difficile colitis, as well as in other infectious enterocolitides and inflammatory bowel disease.

The production of chemokines in general, and IL-8 in particular, is regulated largely at the level of gene transcription. More specifically, the promoter region of chemokine genes carry binding motifs for nuclear regulatory factors and gene transcription is controlled through activation of these regulatory elements. NF-κB is a prime regulator of IL-8 gene transcription. The human IL-8 gene, located on the q12-21 region of chromosome 4, carries an NF-kB binding motif at nuceotides −80 to −70 of its promoter region. NF-KB acts synergistically with other nuclear factors to activate IL-8 gene transcription. An NF-IL6 binding site lies immediately adjacent to the NF-κB site on the IL-8 gene (nucleotides −94 to −81) and in a variety of cells IL-8 secretion is regulated by NF-kB in conjunction with NF-IL6. In gastric cancer cell lines NF-kB and AP-1 (which has a binding site at nucleotides −126 to −120) together up-regulate IL-8 production in response to cytokine stimulation.

The NF-kB family of transcription factors regulate the activation of a wide variety of genes that respond to immune or inflammatory signals. Activation of NF-κB leads to the production of pro-inflammatory and anti-apoptotic proteins. Many genes encoding cytokines, chemokines, and cell surface receptors involved in immune recognition, antigen presentation, and leukocyte adhesion are induced following NF-κB activation. NF-κB activation can also protect cells from undergoing apoptosis in response to DNA damage or cytokine stimulation.

The classical form of activated NF-κB is a heterodimer consisting of one p50 and one p65 subunit. Prior to activation, NF-κB resides in the cytoplasm and must translocate to the nucleus to function. Inactive, cytoplasmic NF-κB exists as a trimer bound to a member of the IκB family of inhibitor proteins (e.g., IκBα, IκBβ, and IκBε), the most well characterized and studied being IκBα. Cellular activation by a variety of stimuli (e.g., C. difficile toxin A, LPS, IL-1, TNFα, or contact with pathogens) results in phosphorylation of IκBα, which is then enzymatically conjugated with ubiquitin marking it for degradation by the 26S proteasome. The active NF-κB dimer is then free to translocate to the nucleus, bind to DNA at κB binding sites, and up-regulate gene transcription.

Pharmacological inhibition of NF-κB would be beneficial in the treatment of both inflammation and neoplasia. In fact, agents that inhibit NF-κB activation, such as glucocorticoids and aspirin, have been used for many years to reduce inflammation in a wide variety of human diseases (e.g., asthma, rheumatoid arthritis, and Crohn's disease). More recently, other NF-kB inhibitors, such as dominant negative IκB proteins have been reported to potentiate the effects of chemotherapy and radiation therapy in the treatment of cancer in animal models.

### SUMMARY OF THE INVENTION

The invention features a novel soluble anti-inflammatory factor (SAIF) characterized as being a compound which has a molecular weight from 500 to 1000 daltons, is secreted from a yeast cell, e.g., a cell of the genus Saccharomyces (e.g., Saccharomyces cerevisiae and Saccharomyces boulardii, e.g., A.T.C.C. Deposit No. MYA-796 and A.T.C.C. Deposit No. MYA-797), is a glycan or a glycopeptide, has SAIF biological activity (e.g., inhibits IκB degradation, inhibits IL-8 production, or inhibits NF-κB activation) in a cell contacted with a pro-inflammatory agent or in a cell of a patient having an inflammatory condition, and is heat stable. SAIF is resistant to proteinases (e.g., proteinase K and chymotrypsin), treatment with a single glycosidase (e.g., each one of α-mannosidase, α-galactosidase, β-galactosidase, and β-N-acetylglucosaminidase), treatment with a mixture of deglycosylases (e.g., PNGase F, O-glycosidase, sialidase, β-galactosidase, glucosaminidase, and endo F1), alkaline phosphatase, DNAse, 2-O-sulfatase, and β-glucuronidase. SAIF is sensitive to a mixture of glycosidases (e.g., the combination of α-mannosidase, β-mannosidase, α-glucosidase, β-glucosidase, α-galactosidase, β-galactosidase, α-L-fucosidase, β-xylosidase, α-N-acetylglucosaminidase, β-N-acetylglucosaminidase, α-N-acetylgalactosaminidase, and β-N-acetylgalactosaminidase) and aryl-sulfatase (contaminated with β-glucuronidase). In a preferred embodiment, SAIF is non-proteinaceous compound.

The invention also provides methods of SAIF production and purification, and methods of using SAIF for the treatment or prevention of an inflammatory disease or disorder, as is described herein below and in the claims.

### Definitions

The term “administration” or “administering” refers to a method of giving a dosage of a pharmaceutical composition comprising a SAIF compound to a subject, e.g., a human, where the method is, e.g., topical, oral, intravenous, intraperitoneal, or intramuscular. The preferred method of administration can vary depending on various factors, e.g., the components of the pharmaceutical composition, site of the potential or actual inflammatory disease and severity of disease.

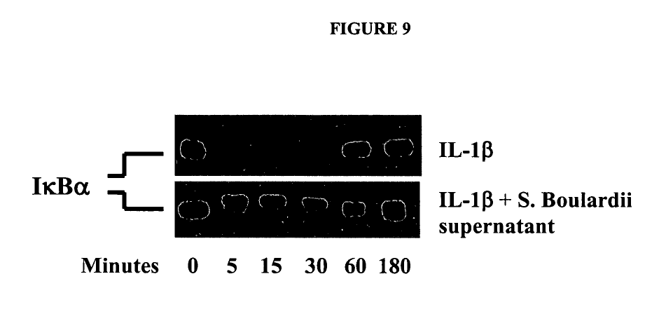

By “inhibits IκB degradation” is meant a compound that is able to reduce or completely prevent the biological breakdown of IκB in a cell that is responding to an inflammatory stimulus, e.g., a cytokine, LPS, or a toxin, when that cell is contacted with the SAIF compound. Preferably, the reduction is by at least 5%, more desirably, by at least 10%, even more desirably, by at least 25%, 50%, or 75%, and most desirably, by 90% or more as determined using the IκB degradation assay described in FIG. 9, when compared to a control lacking a SAIF compound, or any other anti-inflammatory compound.

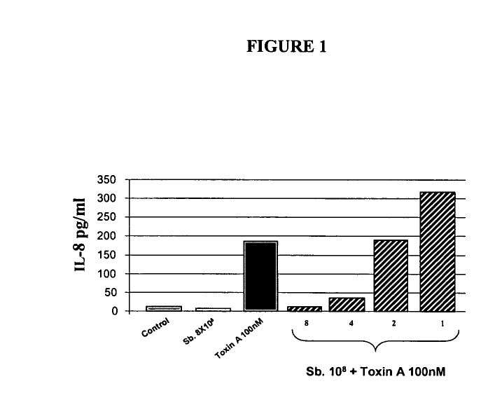

By “inhibits IL-8 production” is meant a compound that is able to reduce or completely prevent the expression and release of interleukin-8 (IL-8) by a cell that is contacted with the SAIF compound in the presence of an inflammatory stimulus, e.g., a cytokine, LPS, or a toxin. Preferably, the reduction is by at least 5%, more desirably, by at least 10%, even more desirably, by at least 25%, 50%, or 75%, and most desirably, by 90% or more as determined using the IL-8 production assays described in FIGS. 1-8, and in the materials and methods section, when compared to a control lacking a SAIF compound or any other anti-inflammatory compound.

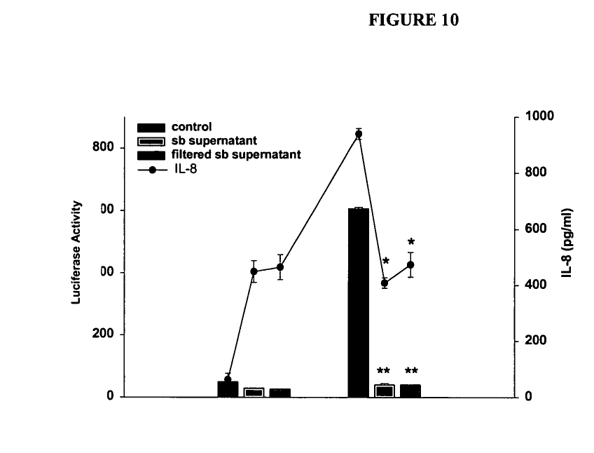

By “inhibits NF-κB activation” is meant a compound that is able to reduce or completely prevent the activation of gene expression mediated by NF-κB by a cell that is contacted with the SAIF compound in the presence of an inflammatory stimulus, e.g., a cytokine, LPS, or a toxin. Preferably, the reduction is by at least 5%, more desirably, by at least 10%, even more desirably, by at least 25%, 50%, or 75%, and most desirably, by 90% or more as determined using the NF-κB activation assay described in FIG. 10, and in the materials and methods section, when compared to a control lacking a SAIF compound or any other anti-inflammatory compound.

By “isolated” is meant a compound of interest (e.g., a SAIF compound) that is in an environment different from that in which the compound naturally occurs. “Isolated” is meant to include compounds that are within samples that are substantially enriched for the compound of interest and/or in which the compound of interest is partially or substantially purified.

By “glycan” is meant any of a diverse class of high-molecular weight carbohydrates formed by the linking together by condensation of monosaccharide, or monosaccharide derivative, units into linear or branched chains, and including homo-polysaccharides (composed of only one type of monosaccharide only) and hetero-polysaccharides (composed of a mixture of different monosaccharide). Found as storage products (e.g. starch and glycogen) and structural components of cell walls (e.g. cellulose, xylans and arabinans), and as components of glycoconjugates.

By “glycopeptide” is meant a compound consisting of carbohydrate linked to a short chain of L- and/or D-amino acids (e.g., ˜1-50 amino acids).

By “glycoprotein” is meant a macromolecule consisting of carbohydrate linked to a protein having a length of greater than 50 amino acids. The carbohydrate is attached to the protein in the form of chains of monosaccharide units attached to specific amino acid residues.

By “pharmaceutically acceptable carrier” is meant a carrier which is physiologically acceptable to the treated mammal while retaining the therapeutic properties of the compound with which it is administered. One exemplary pharmaceutically acceptable carrier is physiological saline. Other physiologically acceptable carriers and their formulations are known to one skilled in the art and described, for example, in Remington 's Pharmaceutical Sciences, (18^{th} edition), ed. A. Gennaro, 1990, Mack Publishing Company, Easton, Pa. incorporated herein by reference.

By “SAIF biological activity” is meant a compound which inhibits at least one of IL-8 production, IkB degradation, or NF-kB activation by at least 10%.

By “SAIF compound” is meant a compound having anti-inflammatory activity that is present in the extract from yeast (e.g., A.T.C.C. Deposit No. MYA-796 and A.T.C.C. Deposit No. MYA-797), and which is characterized as being glycosylated factor (e.g., a glycan or a glycopeptide) having a molecular weight of less than 1,000 daltons, heat stable (e.g., retains anti-inflammatory activity after exposure to 100° C. for 5 minutes), and the ability to inhibit IL-8 production, IκB degradation, and NF-κB activation in a cell that has been exposed to an inflammatory stimulus. In addition to being glycosylated, a SAIF compound of the invention may also be sulfated. A SAIF compound is resistant to degradation by proteinases (e.g., proteinase K and chymotrypsin), single glycosidases (e.g., each one of α-mannosidase, α-galactosidase, β-galactosidase, and β-N-acetylglucosaminidase), deglycosylases (e.g., PNGase F, O-glycosidase, sialidase, β-galactosidase, glucosaminidase, and endo F1), alkaline phosphatase, DNAse, 2-O-sulfatase, and β-glucuronidase.

By “substantially pure” is meant that a compound (e.g., a SAIF compound) has been separated from at least 60% to 75% or more of the components (e.g., proteins) that naturally accompany it. Preferably, a SAIF compound of the invention is substantially pure when it is separated from at least about 85 to 90% of the components that naturally accompany it, more preferably at least about 95%, and most preferably about 99%. Normally, purity is measured on a chromatography column, polyacrylamide gel, or by HPLC analysis.

By “therapeutically effective amount,” we mean the amount of CD39 polypeptide needed to produce a substantial clinical improvement. Optimal amounts will vary with the method of administration, and will generally be in accordance with the amounts of conventional medicaments administered in the same or a similar form.

By “treating or preventing” is meant administering a pharmaceutical composition comprising a SAIF compound for prophylactic and/or therapeutic purposes. To “prevent disease” refers to prophylactic treatment of a patient who is not yet ill, but who is susceptible to, or otherwise at risk of, an inflammatory disease or disorder. To “treat disease” or use for “therapeutic treatment” refers to administering a SAIF compound to a patient already suffering from an inflammatory disease to ameliorate the disease and improve the patient's condition. Thus, in the claims and embodiments, treating is the administration to a subject, e.g., a human, either for therapeutic or prophylactic purposes.

### BRIEF DESCRIPTION OF THE INVENTION

FIG. 1 is a graph showing that SAIF induces a dose-dependent inhibition of C. difficile toxin A-mediated IL-8 production by THP-1 cells. THP-1 cells (5×10⁵/mL) were co-incubated with S. boulardii alone (8×10⁸ cfu/mL), purified C. difficile toxin A alone (100 nM), or with varying concentrations of S. boulardii (1 to 8×10⁸ cfu/mL) together with toxin A (100 nM) for 5 hours after which IL-8 levels in the conditioned media were measured by ELISA.

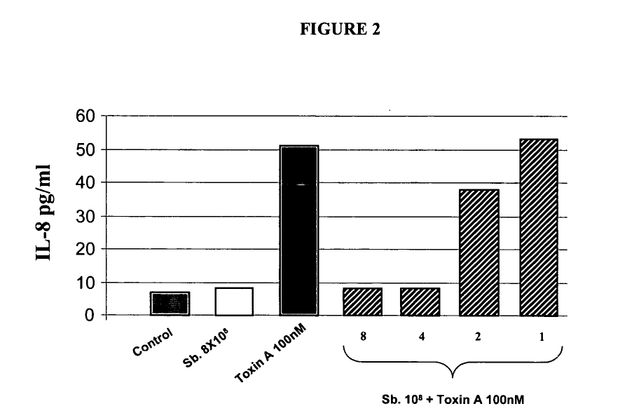

FIG. 2 is a graph showing SAIF-mediated inhibition of toxin A-induced IL-8 production by human monocytes. In the absence of SAIF, C. difficile toxin A activates IL-8 production in non-transformed human peripheral blood monocytes. Human peripheral blood monocytes (2×10⁵/ml) were incubated with S. boulardii (1 to 8×10⁸ cfu/mL) and/or purified C. difficile toxin A (100 nM) for 5 hours after which IL-8 levels in the conditioned media were measured by ELISA.

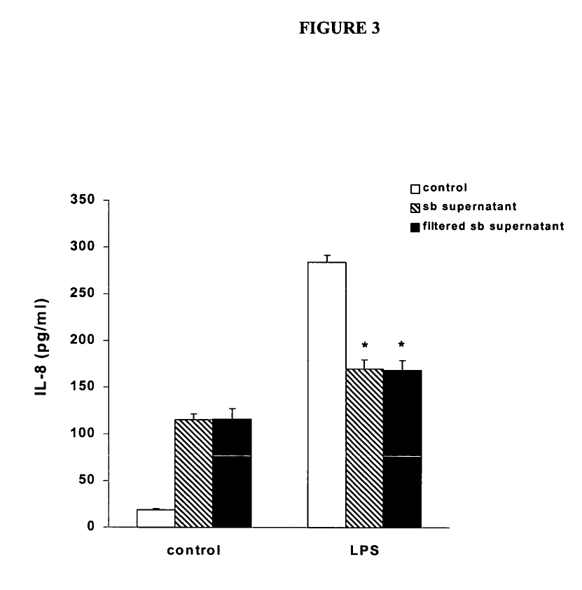

FIG. 3 is a graph showing that SAIF is a soluble factor that mediates an inhibitory effect on IL-8 production in the presence of lipopolysaccharide (LPS). One gram of lyophilized S. boulardii was incubated in RPMI growth medium for 24 hours at 37° C. The suspension was then centrifuged at 7,400 rpm for 15 minutes and the supernatant collected (Sb supernatant). Filtered Sb supernatant was produced by passing the supernatant through a 0.22 μm filter (Fisher Scientific, Agawam, Mass.). THP-1 monocytic cells (100 μL; final concentration 5×10⁵/mL) were co-incubated with 100 μL S. boulardii supernatant or filtered S. boulardii supernatant, in the presence or absence of purified LPS (100 ng/mL, from Escherichia coli 055:B5, Sigma) for 5 hours after which IL-8 levels in the conditioned media were measured by ELISA. Both the S. boulardii supernatant and the filtered S. boulardii supernatant inhibited IL-8 production by LPS-stimulated THP-1 cells (ANOVA, p<0.0001. * denotes p<0.001 compared to LPS alone by Bonferroni test), indicating that SAIF is a soluble factor.

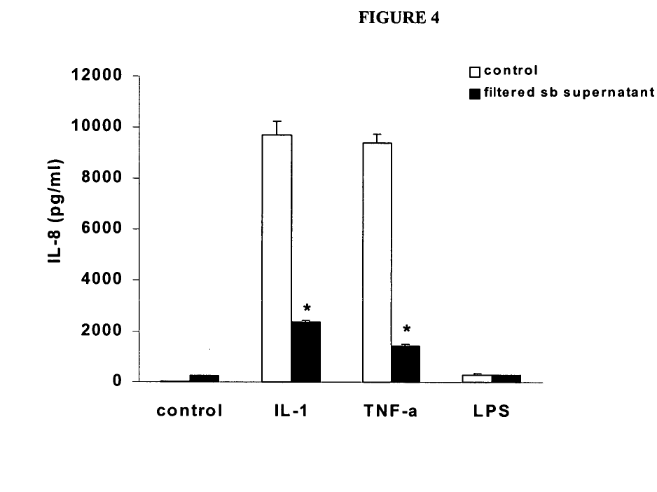

FIG. 4 is a graph showing the inhibitory effects of SAIF on intestinal epithelial cells. HT-29 human transformed intestinal epithelial cells were seeded onto 96 well plates. After reaching confluency the cells were serum starved overnight and then stimulated with IL-1β (10 ng/mL), TNF-α (10 ng/mL), or LPS (100 ng/mL), in the presence or absence of filtered S. boulardii supernatant. After 12 hours incubation the HT-29 cell conditioned media were collected and IL-8 protein levels were measured by ELISA. The filtered S. boulardii supernatant inhibited IL-8 production in both IL-1- and TNF-α-stimulated HT-29 cells (* denotes p<0.001 by Student t-test when compared to IL-1 or TNF-α stimulation alone. As expected LPS resulted in minimal activation of IL-8 production in HT-29 intestinal epithelial cells.

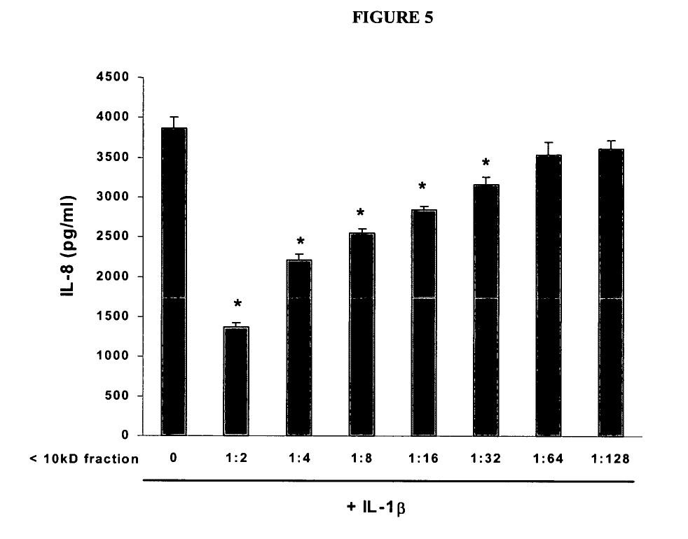

FIG. 5 is a graph showing the dose-dependent inhibition of IL-8 production in IL-1β-stimulated HT-29 cells by SAIF. Confluent monolayers of HT-29 cells were stimulated with IL-1β (10 ng/mL) alone, or in the presence of serial two fold dilutions of filtered S. boulardii supernatant that had been fractionated through a 10 kD filter (Millipore, Bedford, Mass.). After a 12 hour incubation, HT-29 cell culture supernatants were collected and IL-8 levels were measured by ELISA. Data are shown for serial 2 fold dilutions of the filtered S. boulardii supernatant from 1:2 to 1:128 volume/volume dilution in HT-29 culture medium. The <10 kD fraction of filtered S. boulardii supernatant containing SAIF inhibited IL-8 production by IL-1-stimulated HT-29 cells in a dose dependent manner (ANOVA, p<0.0001. * denotes p<0.001 compared to IL-1 stimulation alone by Bonferroni test).

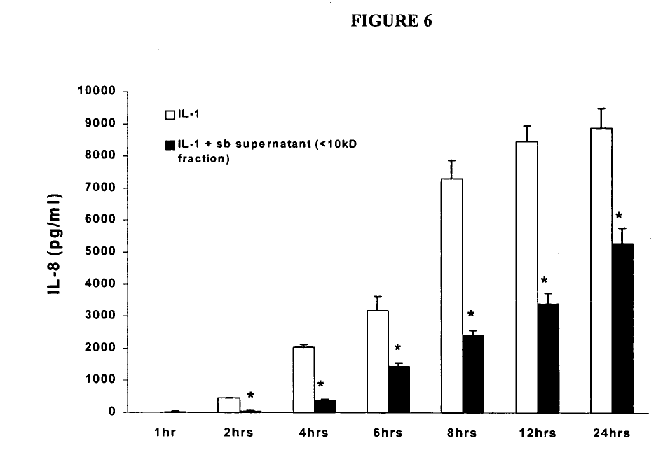

FIG. 6 is a graph showing SAIF-mediated inhibition of IL-8 production over time in HT-29 cells stimulated by IL-1β. HT-29 cells were stimulated with IL-1β (10 ng/mL) in the presence or absence of the <10 kD fraction of filtered S. boulardii culture supernatant. After incubation periods of 1 to 24 hours, HT-29 cell conditioned media were collected and IL-8 levels were measured by ELISA. The filtered S. boulardii supernatant significantly inhibited IL-8 production by IL-1β-stimulated HT-29 cells at every time point examined between 2 and 24 hours (* denotes p<0.01 compared to IL-1 stimulation alone at each respective time point, Student t test).

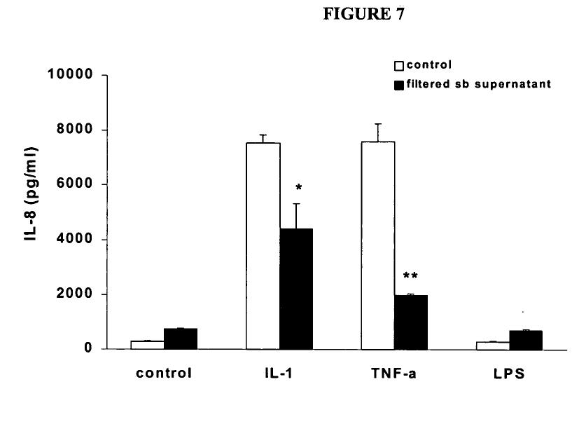

FIG. 7 is a graph showing SAIF-mediated inhibition of IL-8 production in IL-1β- and TNF-α-stimulated AGS gastric epithelial cells. AGS human transformed gastric epithelial cells were seeded onto 96 well plates. After reaching confluency the cells were stimulated with IL-1β (10 ng/mL), TNF-α (10 ng/mL), or LPS (10 ng/mL) in the presence or absence of filtered S. boulardii supernatant. After 12 hours the conditioned media were collected and IL-8 protein levels were measured by ELISA. The filtered S. boulardii supernatant inhibited IL-8 production in both IL-1β- and TNF-α-stimulated AGS cells (* denotes p=0.01 compared to IL-1 alone, ** denotes p<0.001 compared to TNF alone, t test). As expected LPS resulted in minimal activation of IL-8 production in AGS gastric epithelial cells.

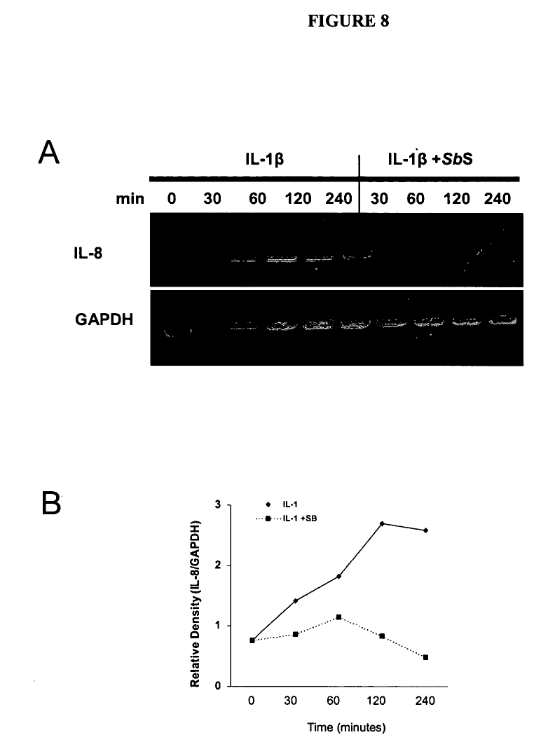

FIGS. 8A and 8B demonstrate that SAIF inhibits IL-1β-mediated increases in IL-8 mRNA levels in HT-29 colonic epithelial cells. FIG. 8A is a photograph of an ethidium bromide-labeled gel showing that HT-29 cells treated with IL-1β alone show an early and sustained increase in steady state IL-8 mRNA levels consistent with upregulation of IL-8 gene expression. This increase in IL-8 mRNA levels was inhibited by treatment with S. boulardii supernatant. HT-29 cells were seeded in 6 well plates and stimulated with IL-1β (10 ng/mL) in the presence or absence of filtered S. boulardii supernatant. Cells were harvested at 0 min, 30 min, 1 h, 2 h, and 4 h, and total RNA was extracted. Two micrograms of RNA was then reverse transcribed to yield complementary DNA (cDNA). The undiluted cDNA solution was subsequently subjected to PCR amplification for IL-8 and GAPDH, using appropriate primers. The PCR products were analyzed by electrophoresis through 1.2% agarose gels containing 100 ng/mL ethidium bromide. The DNA bands corresponding to IL-8 and GAPDH were visualized using an ultraviolet transilluminator (Biorad) and their density was calculated using the Quantity One software (Biorad). FIG. 8B is a graph showing quantifying IL-8 mRNA levels in IL-1β-stimulated HT-29 colonic epithelial cells in the presence or absence of S. boulardii supernatant. IL-8 mRNA levels (as determined by RT-PCR) at the indicated time points are expressed as a ratio IL-8 band density versus GAPDH density.

FIG. 9 is a photograph showing a western blot of IκBα using an anti-IκBα antibody demonstrating that SAIF prevents IκB degradation following cellular activation. THP-1 cells were seeded in 10 mm tissue culture dishes at a concentration of 8×10⁵ cells/mL and stimulated with IL-1β (10 ng/mL) or IL-1β plus filtered S. boulardii culture supernatant for the indicated time periods. Cytoplasmic extracts were then prepared and subjected to Western blotting using an anti-IκBα antibody. The S. boulardii supernatant prevented IL-1β-induced IκBα degradation (5 to 30 minute time points). IκB degradation is a critical step towards NF-κB activation and nuclear translocation. Thus the ability of SAIF to prevent IκB degradation provides a potential mechanism for its anti-inflammatory effect.

FIG. 10 is a graph showing a reduction in LPS-induced NF-κB-reporter gene activation in THP-1 cells in the presence of SAIF. THP-1 cells (2×10⁷/mL) were transiently transfected with an NF-κB-responsive luciferase reporter gene construct using the DEAE-dextran procedure. Briefly, 2×10⁷ THP-1 cells were suspended in 1 mL prewarmed Tris-buffered saline and incubated for 10 minutes at 37° C. with 80 μg DEAE-dextran (Pharmacia). THP-1 cells were then transfected with 5 μg DNA of the luciferase NF-kB reporter plasmid. Transfection was stopped by adding 25 mL Tris-buffered saline. After washing, cells were cultured for 48 hours before stimulation. After stimulation with S. boulardii culture supernatant and/or purified LPS (100 ng/mL) for 5 hours, THP-1 cells (8×10⁶ cells per stimulus) were washed in PBS. The cell lysis and luciferase assay was performed using the Luciferase Assay System (Promega Corp.), according to the instructions ofthe manufacturer. Culture supernatants were also collected for IL-8 protein measurement by ELISA. Both the S. boulardii supernatant and filtered Sb supernatant completely prevented LPS-induced NF-κB-reporter gene activation in THP-1 cells (** denotes p<0.001 compared to control LPS stimulation alone, * denotes p<0.05 compared to LPS stimulation alone).

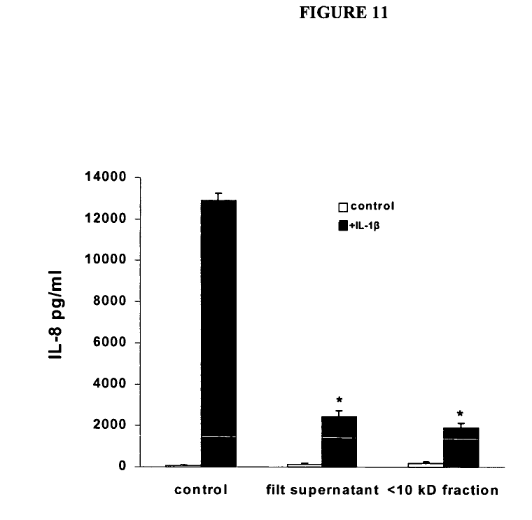

FIG. 11 is a graph showing that the inhibitory activity of the S. boulardii culture supernatant was retained in the <10 kD fraction. S. boulardii supernatant was produced as described in the legend to FIG. 3, the pH neutralized (to pH 7.0) with NaOH (35 mM) and filtered through a 0.22 μm filter (Fisher Scientific, Agawam, Mass.), followed by fractionation through a 10 kD filter (Millipore, Bedford, Mass.). Data shown are IL-8 protein levels (pg/mL) in HT-29 cell conditioned media following stimulation of the HT-29 cells with IL-1β (10 ng/mL). Inhibitory activity was consistently retained in the <1 kD fraction (as shown in FIG. 11, * denotes P<0.001 compared to IL-1 alone by Student's t test). The finding that the inhibitory factor has a molecular mass of <10 kDa was further supported by dialysis of the supernatant against PBS, pH: 7.4, through a 12 kD dialysis membrane which resulted in loss of inhibitory activity.

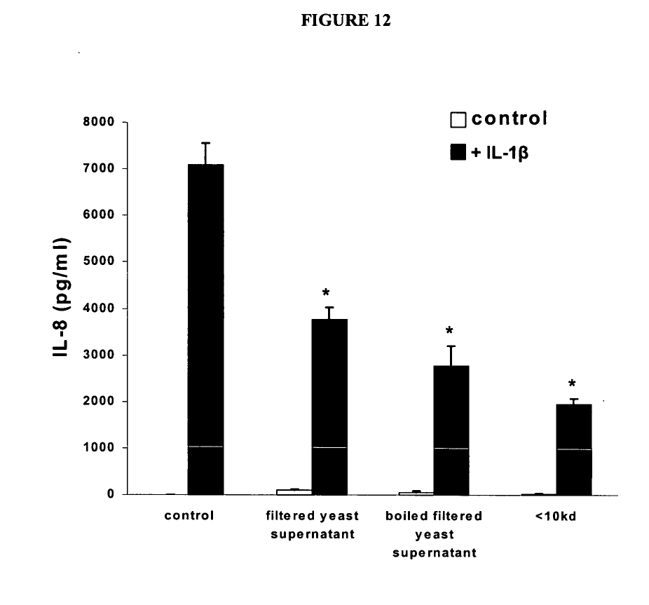

FIG. 12 is a graph showing the activity of SAIF following heat treatment at 100° C. for 5 minutes. Data are shown as IL-8 protein levels (pg/mL) in HT-29 cell conditioned media (p<0.001 by ANOVA; * denotes p<0.001 compared to IL-1 alone (Bonferroni)). There is no significant difference in the inhibitory activity between filtered yeast supernatant and boiled filtered yeast supernatant (p>0.05).

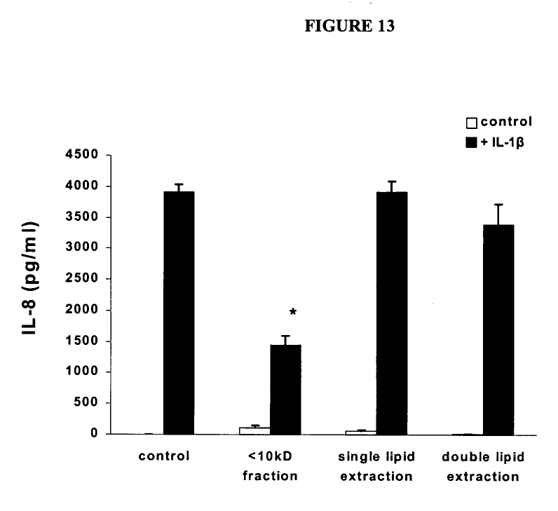

FIG. 13 is a graph showing the activity of SAIF following lipid extraction from the <10 kD fraction of the filtered S. boulardii supernatant by liquid-liquid extraction using 6 volumes of chloroform-methanol (2:1, v/v) in a glass tube. After centrifugation at 800×g for 3 min, the resulting lower phase (organic phase) was aspirated and transferred to a separate tube. The organic solvents were then evaporated in the presence of N₂ and the dried material was reconstituted in HT-29 media by sonication. In some cases the organic phase was subjected to a second cycle of the same procedure (double lipid extraction). The <10 kD fraction of the S. boulardii supernatant remains active following lipid extraction (p<0.001 by ANOVA; * denotes p<0.001 compared to IL-1 alone by Bonferroni test). In contrast, lipids extracted from the <10 kD fraction do not show any inhibitory activity (p>0.05 compared to IL-1 alone). Data shown are IL-8 protein levels (pg/mL) in HT-29 cell conditioned media.

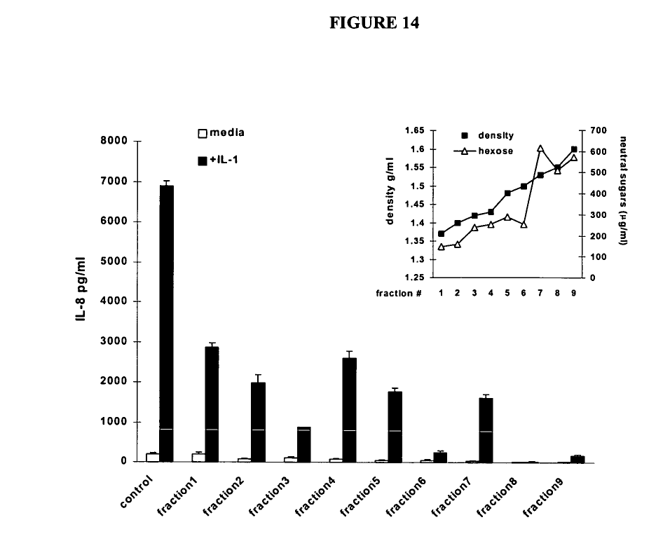

FIG. 14 is a graph showing that the heaviest fractions of the S. boulardii supernatant contain the greatest inhibitory activity against IL-8 production, indicating that SAIF is a dense, heavily glycosylated glycan, glycopeptide, or other glycosylated compound. FIG. 14 (inset) shows that the S. boulardii supernatant fractions with the highest IL-8 production inhibition activity have a high level of neutral sugars.

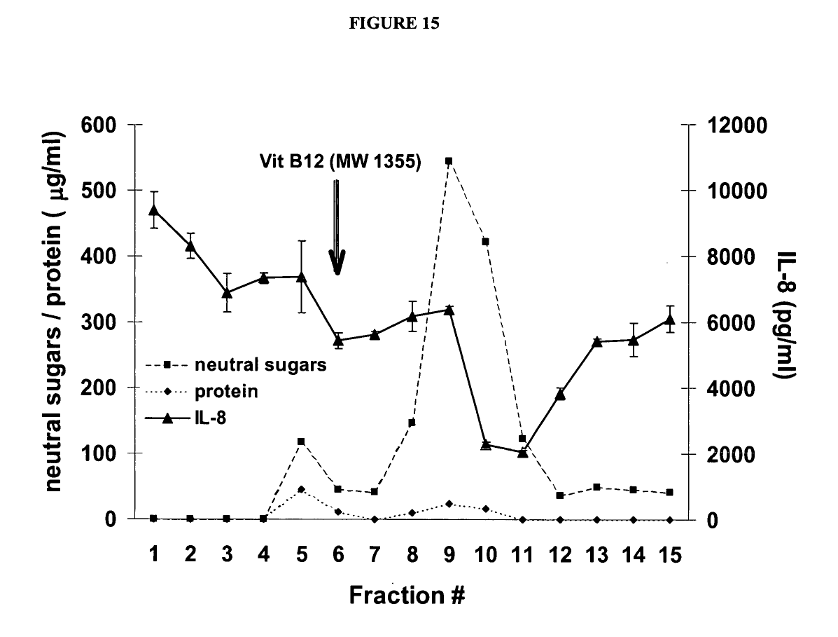

FIG. 15 is a graph showing that SAIF is a small dense glycan/glycopeptide containing high levels of hexose. Following cesium chloride gradient separation, the more dense fractions (7, 8 and 9; see FIG. 14) were pooled and further separated through a Biogel P-30 column. Hexose (neutral sugars, shown as μg/mL) and protein levels (shown as μg/mL) were measured in the resulting 18 fractions that were also tested for their ability to inhibit IL-8 protein production in IL-1β-stimulated HT-29 monolayers. The fractions that contained the highest levels of hexose (neutral sugars) and protein (fractions 10 and 11) were active in inhibiting IL-8 production (ANOVA, p<0.001. Bonferroni tests for fractions 10, 11: p<0.05 for each, compared to control (i.e., IL-1β stimulation alone); for all other fractions p>0.05), and had measurable levels of neutral sugars and protein by the phenol-sulfuric acid and bicinchoninic acid protein assay (BCA; Pierce Laboratories, Rockford, Ill.) methods, respectively. Vitamin B12, used as a molecular weight marker, was eluted under the same conditions at fraction 6, indicating that the active substance is <1 kD.

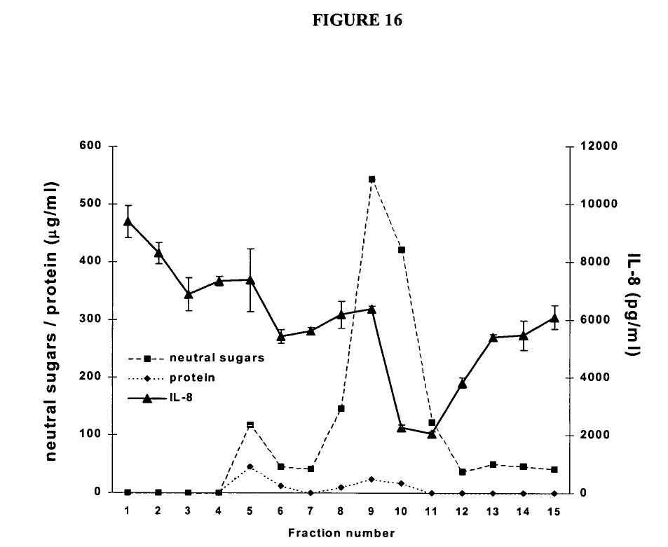

FIG. 16 is a graph showing that SAIF has a molecular weight of <1 kD. Following cesium chloride gradient separation, the more dense fractions (fractions 7, 8, and 9; see FIG. 14) were pooled and eluted through a Biogel P-2 column (fractionation range 100-1800 Daltons) in order to achieve better separation than the Biogel P-30 column (see FIG. 15). Hexose (neutral sugars, shown as μg/mL) and protein levels (shown as μg/mL) were measured in the resulting 15 fractions that were also tested for their ability to inhibit IL-8 protein production in IL-1β-stimulated HT-29 monolayers. Fractions 10 and 11 potently inhibited IL-8 production (ANOVA, p=0.0003; Bonferroni test for fractions 10 and 11; p<0.05 for each compared to IL-1β stimulation alone; p>0.05 for all other fractions). Under the same conditions, vitamin B12 (molecular weight 1,355 Daltons) eluted from the P-2 column in a peak with maximum at fraction # 6. SAIF elutes in fractions 10, 11, and 12, and therefore, has a molecular weight of less than 1,000 Da.

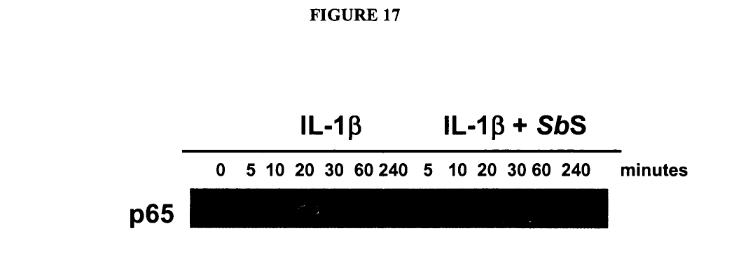

FIG. 17 is a photograph showing a western blot of the nuclear levels of p65, as detecting by using an anti-p65 antibody. THP-1 cells were stimulated with IL-1β (10 ng/ml), and nuclear extracts were prepared at the indicated time points and subjected to western blotting.

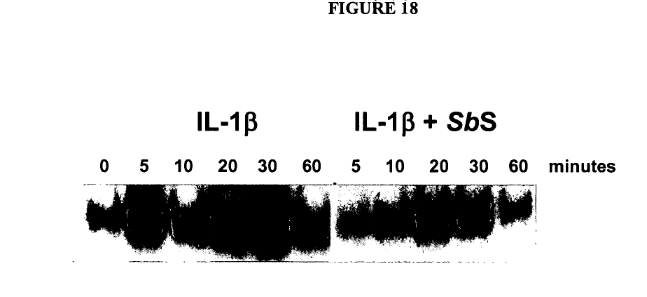

FIG. 18 is a photograph showing NF-κB-DNA binding activity, which was examined by electrophoretic mobility shift assay (EMSA) using a ³²P-labeled probe corresponding to the consensus NF-κB binding site. Electrophoretic mobility shift assay (EMSA) is performed by taking nuclear extracts from THP-1 cells that were stimulated with IL-1β (10 ng/ml), either alone or in the presence of S. boulardii supernatant, as described above. The consensus NF-κB binding site was synthesized as a double stranded oligonucleotide by Operon (San Francisco, Calif.), and was end labeled with (³²P) dCTP by Klenow DNA Polymerase (New England Biolabs; Beverly, Mass.). The resulting probe was purified on a Quick-Sep Column (Isolab, Inc.; Akron, Ohio) and percent binding was calculated. EMSA experiments were performed as previously described (see, e.g., Simeonidis et al., PNAS USA 96:49-54, 1999, and Merika et al., Mol. Cell 1:277-287, 1998). Briefly, in the binding mixture, 6 μg of nuclear proteins, 2 μl of radioactive probe (80,000-100,000 cpm), binding buffer, and water were added to a final volume of 20 μl. The binding buffer consisted of 50 mM MgCl₂, 340 mM KCl with 3 μg/μl poly dI-dC in a 5:3 ratio with a secondary buffer containing 0.1 mM EDTA (pH 8), 40 mM KCl, 25 mM Hepes (pH 7.6), 8% Ficoll and 1 mM of DTT. Certain reactions also contained 100-fold excess of the specific unlabeled consensus oligonucleotide in order to determine the specificity of the binding reaction. The binding mixtures were incubated for 15 minutes in room temperature and then analyzed on non-denaturing 6% polyacrylamide gels in Tris-Boric-EDTA (pH 7.4). Gels were run for approximately 3 hours, vacuum-dried, exposed to X-ray film (Kodak; Rochester, N.Y.) and then developed.

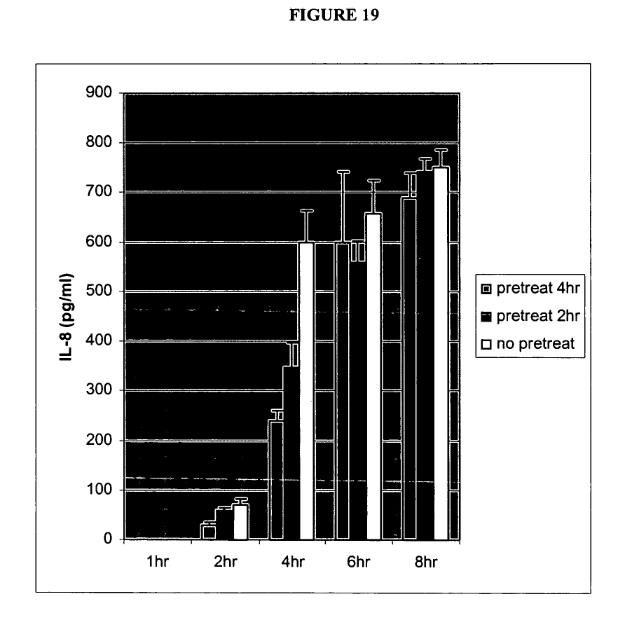

FIG. 19 is a graph showing that pretreatment of HT29 colonic epithelial cells with SAIF for 2 or 4 hours causes a reversible inhibition of IL-1-mediated IL-8 gene expression.

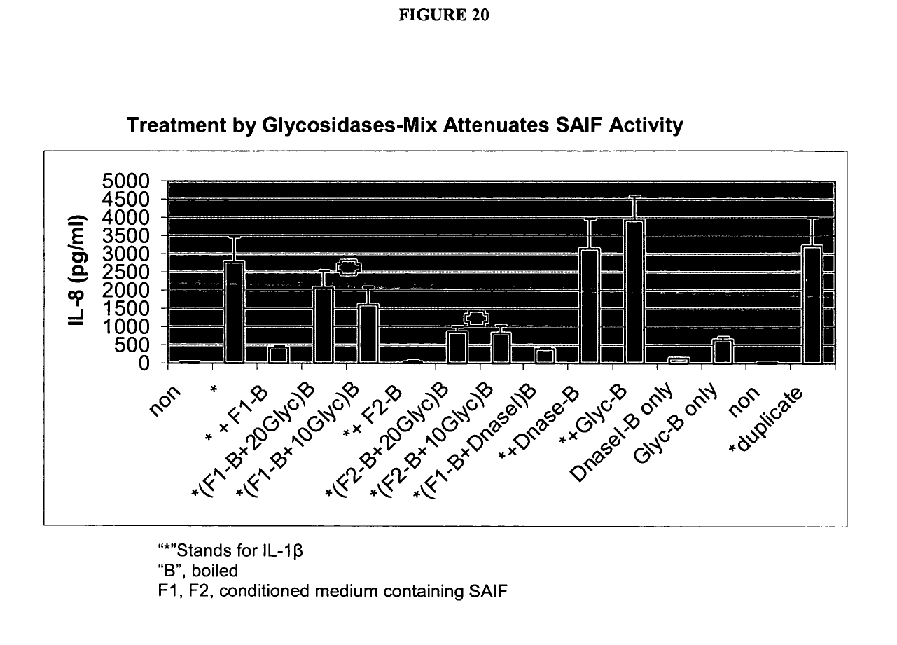

FIG. 20 is a graph showing that treatment of conditioned medium containing SAIF with a mixture of Glycosidases attenuates SAIF activity.

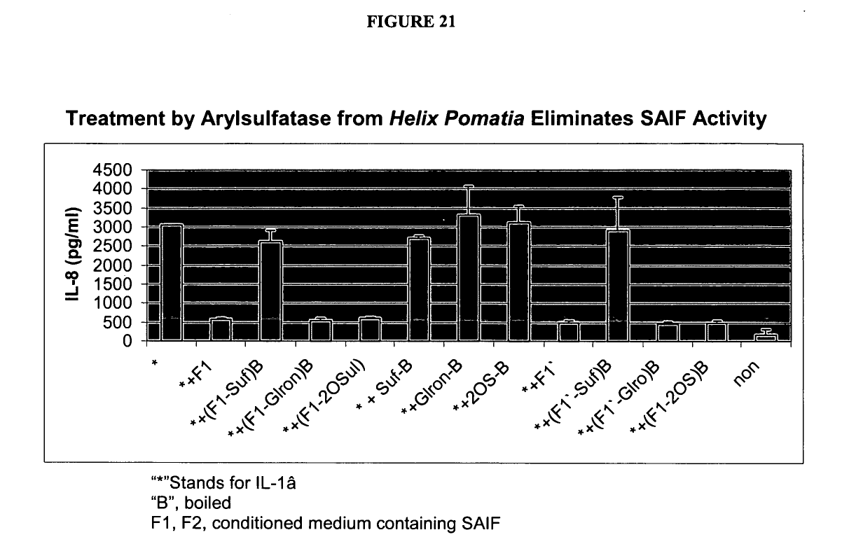

FIG. 21 is a graph showing that treatment of conditioned medium containing SAIF with arylsulfatase from Helix pomatia eliminates SAIF activity.

### DETAILED DESCRIPTION

We have discovered a novel soluble anti-inflammatory factor (SAIF) which inhibits the expression of the pro-inflammatory chemokine IL-8 by inhibiting the degradation of IκB, thereby preventing NF-κB-regulated gene expression. SAIF can be produced in yeast (e.g., Saccharomyces boulardii; ATCC No. MYA-796 and MYA-797, ATCC, P.O. Box 1549, Manassas, Va. 20108; see, also McCullough et al., J. Clin. Microbiol. 36:2613-2617, 1998), and isolated from the supernatant following secretion of SAIF into the culture medium.

Furthermore, a SAIF compound can be administered to a subject in need thereof for the prevention or treatment of inflammatory diseases or disorders, such as those that occur in gastrointestinal injury and inflammatory bowel disease. Specific diseases or disorders include, e.g., Crohn's disease, ulcerative proctitis, ulcerative colitis, and microscopic colitis. In addition, a SAIF compound can be administered to treat or prevent acute or chronic gastrointestinal injury and inflammation caused by infectious agents, such as bacterial, viral, or parasitic agents, or toxin-mediated inflammation.

Inflammation that occurs at extraintestinal sites, including the skin and musculoskeletal system due to, e.g., psoriasis, dermatitis, rheumatoid arthritis, or degenerative joint disease, can also be treated by administration of a SAIF compound. Because of its ability to prevent NF-kB-regulated gene expression, a SAIF compound may also be a valuable adjunct to chemotherapeutic agents used in the treatment of neoplastic disorders. A SAIF compound can also be used in the treatment of inflammatory conditions, such as asthma.

The present invention also includes pharmaceutical compositions and formulations which include a SAIF compound, or analogue thereof. The pharmaceutical compositions of the present invention may be administered in any number of ways depending upon whether local or systemic treatment is desired and upon the area to be treated. Administration may be topical (including ophthalmic, vaginal, rectal, intranasal, transdermal), oral, or parenteral. Parenteral administration includes intravenous drip, continuous infusion, subcutaneous, intraperitoneal or intramuscular injection, pulmonary administration, e.g., by inhalation or insufflation, or intrathecal or intraventricular administration.

Methods well known in the art for making formulations are found, for example, in Remington's Pharmaceutical Sciences (18th edition), ed. A. Gennaro, 1990, Mack Publishing Company, Easton, Pa. Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the polypeptides of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

Compositions for rectal or vaginal administration are desirably suppositories which may contain, in addition to active substances, excipients such as coca butter or a suppository wax. Compositions for nasal or sublingual administration are also prepared with standard excipients known in the art. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops or spray, or as a gel.

The amount of active ingredient in the compositions of the invention can be varied. One skilled in the art will appreciate that the exact individual dosages may be adjusted somewhat depending upon a variety of factors, including the compound being administered, the time of administration, the route of administration, the nature of the formulation, the rate of excretion, the nature of the subject's conditions, and the age, weight, health, and gender of the patient. Generally, dosage levels of between 0.1 μg/kg to 100 mg/kg of body weight are administered daily as a single dose or divided into multiple doses. Desirably, the general dosage range is between 250 μg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

The SAIF compound of the invention can be administered in a sustained release composition, such as those described in, for example, U.S. Pat. No. 5,672,659 and U.S. Pat. No. 5,595,760. The use of immediate or sustained release compositions depends on the type of condition being treated. If the condition consists of an acute or over-acute disorder, a treatment with an immediate release form will be desired over a prolonged release composition. Alternatively, for preventative or long-term treatments, a sustained released composition will generally be desired.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Materials and Methods

S. Boulardii Inhibits Clostridium Difficile Toxin A-Induced IL-8 Production by THP-1 Human Monocytic Cells.

C. difficile toxin A activates an inflammatory response in THP-1 human monocytic cells as evidenced by increased production and release of the pro-inflammatory chemokine IL-8. We first examined the effects of S. boulardii on toxin A-mediated THP-1 cell activation.

THP-1 cells (5×10⁵/mL) were co-incubated with S. boulardii alone (8×10⁸ cfu/mL), purified C. difficile toxin A alone (100 nM), or with varying concentrations of S. boulardii (1 to 8×10⁸ cfu/mL) together with toxin A (100 nM) for 5 hours after which IL-8 levels in the conditioned media were measured by ELISA.

As shown in FIG. 1, S. boulardii induced a profound, dose-dependent inhibition of toxin A-Induced IL-8 production by the THP-1 cells.

S. Boulardii Inhibits C. Difficile Toxin A-induced IL-8 Production by Human Peripheral Blood Monocytes.

C. difficile toxin A also activates IL-8 production in non-transformed human peripheral blood monocytes. We therefore examined whether S. boulardii could also inhibit toxin A-mediated activation of human monocytes.

Human peripheral blood monocytes (2×10⁵/mL) were incubated with S. boulardii (1 to 8×10⁸ cfu/mL) and/or purified C. difficile toxin A (100 nM) for 5 hours after which IL-8 levels in the conditioned media were measured by ELISA.

As shown in FIG. 2, S. boulardii completely inhibited toxin A-induced IL-8 production by human monocytes. As for THP-1 cells this effect was dose-dependent within the range of 1 to 4×10⁸ cfu of S. boulardii per mL.

S. Boulardii Culture Supernatant Inhibits IL-8 Production by LPS Stimulated THP-1 Cells.

Having shown that S. boulardii can block IL-8 production in human monocytes and THP-1 cells exposed to C. difficile toxin A, we asked whether this anti-inflammatory effect could attenuate monocyte responses to other bacterial products. Therefore, we examined whether S. boulardii alters monocyte IL-8 production in responses to bacterial lipopolysaccharide (LPS or endotoxin). LPS is known to be a potent stimulus for monocyte and macrophage activation.

To determine whether the inhibitory effect of S. boulardii was mediated by a soluble factor, we prepared and filtered a S. boulardii supernatant. One gram of lyophilized S. boulardii was incubated in RPMI growth medium for 24 hours at 37° C. The suspension was then centrifuged at 7,400 rpm for 15 minutes and the supernatant collected (Sb supernatant). Filtered Sb supernatant was produced by passing the supernatant through a 0.22 μm filter (Fisher Scientific, Agawam, Mass.).

THP-1 monocytic cells (100 μL; final concentration 5×10⁵/ml) were co-incubated with 100 μL S. boulardii supernatant or filtered S. boulardii supernatant, in the presence or absence of purified LPS (100 ng/mL, from Escherichia coli (055:B5; Sigma) for 5 hours, after which IL-8 levels in the conditioned media were measured by ELISA.

As shown in FIG. 3, both the S. boulardii supernatant and the filtered S. boulardii supernatant inhibited IL-8 production by LPS-stimulated THP-1 cells (ANOVA, p<0.0001. * denotes p<0.001 compared to LPS alone by Bonferroni test).

S. Boulardii Supernatant Inhibits IL-8 Production by IL-1β or TNF-α Stimulated HT-29 Colonic Epithelial Cells.

We next examined whether S. boulardii supernatant showed similar inhibitory effects on IL-8 production in intestinal epithelial cells. HT-29 human transformed intestinal epithelial cells were seeded onto 96 well plates. After reaching confluency, they were serum starved overnight and then stimulated with IL-1β (10 ng/mL), TNF-α (10 ng/mL), or LPS (100 ng/mL) in the presence or absence of filtered S. boulardii supernatant. After 12 hours incubation the HT-29 cell conditioned media were collected and IL-8 protein levels were measured by ELISA.

As shown in FIG. 4, the filtered S. boulardii supernatant inhibited IL-8 production in both IL-1β- and TNF-α-stimulated HT-29 cells (* denotes p<0.001 by Student t-test when compared to IL-1 or TNF stimulation alone). As expected, LPS resulted in minimal activation of IL-8 production in HT-29 intestinal epithelial cells.

Filtered S. Boulardii Supernatant Inhibits IL-8 Production by IL-1β Stimulated HT-29 Cells (Dose Response).

Confluent monolayers of HT-29 cells were stimulated with IL-1β (10 ng/mL) alone or in the presence of serial two fold dilutions of filtered S. boulardii supernatant that had been fractionated through a 10 kD filter (Millipore, Bedford, Mass.). After 12 hours incubation HT-29 cell culture supernatants were collected and IL-8 levels were measured by ELISA. Data are shown for serial 2 fold dilutions of the filtered S. boulardii supernatant from 1:2 to 1:128 volume/volume dilution in HT-29 culture medium.

As shown in FIG. 5, the <10 kD fraction of filtered S. boulardii supernatant inhibited IL-8 production by IL-1-stimulated HT-29 cells in a dose dependent manner (ANOVA, p<0.0001. * denotes p<0.001 compared to IL-1 stimulation alone by Bonferroni test).

S. Boulardii Supernatant Inhibits IL-8 Production by IL-1β Stimulated HT-29 Cells (Time Course).

HT-29 cells were stimulated with IL-1β (10 ng/mL) in the presence or absence of the <10 kD fraction of filtered S. boulardii culture supernatant. After incubation periods of 1 to 24 hours the HT-29 cell conditioned media were collected and IL-8 levels were measured by ELISA.

As shown in FIG. 6, the filtered S. boulardii supernatant significantly inhibited IL-8 production by IL-1β-stimulated HT-29 cells at every time point examined between 2 and 24 hours (* denotes p<0.01 compared to IL-1 stimulation alone at each respective time point, Student t test).

S. Boulardii Culture Supernatant Inhibits IL-8 Production by IL-1β or TNF-α Stimulated AGS Gastric Epithelial Cells.

AGS human transformed gastric epithelial cells were seeded onto 96 well plates. After reaching confluency they were stimulated with IL-1β (10 ng/mL), TNF-α (10 ng/mL), or LPS (10 ng/mL) in the presence or absence of filtered S. boulardii supernatant. After 12 hours the conditioned media were collected and IL-8 protein levels were measured by ELISA.

As shown in FIG. 7, the filtered S. boulardii supernatant inhibited IL-8 production in both IL-1β- and TNF-α-stimulated AGS cells (* denotes p=0.01 compared to IL-1β alone, ** denotes p<0.001 compared to TNF-αalone, t test). As expected, LPS resulted in minimal activation of IL-8 production in AGS gastric epithelial cells.

S. Boulardii Culture Supernatant does not Affect THP-1 or HT-29 Cell Viability.

After 5 hours exposure to S. boulardii supernatant, THP-1 cells were examined morphologically and by flow cytometry after the addition of propidium iodide (10 μg/mL). No changes were observed by either method between control cells and cells exposed to Sb supernatant (unfiltered, filtered, and <10 kD fraction).

The viability of HT-29 cells was assessed after 24 hrs exposure to S. boulardii culture supernatant by the MTS (3-[4,5-dimethylthiazol-2-yl-5]-[3-carboxymethoxyphenyl]-2-[4-sulfophenyl]-2H tetrazolium) cell proliferation assay, performed according to the manufacturer's instructions (Promega, Madison, Wis.). No differences in HT-29 cell viability were found in cells incubated with S. boulardii culture supernatant as compared to control. This was further confirmed in parallel experiments where cells exposed to S. boulardii culture supernatant for 12 hours were subsequently stimulated with IL-1β. IL-8 protein level measured in condition medium by ELISA was found to be similar to that produced by cells not previously exposed to S. boulardii culture supernatant, indicating that the S. Boulardii culture supernatant does not affect cell viability or function.

S. Boulardii Culture Supernatant Blocks IL-1β-Mediated Increases in IL-8 mRNA Levels in HT-29 Colonic Epithelial Cells.

HT-29 cells were seeded in 6 well plates and stimulated with IL-1β (10 ng/mL) in the presence or absence of filtered S. boulardii supernatant. Cells were harvested at 30 min, 1 h, 2 h and 4 h and total RNA was extracted. Two micrograms of RNA was then reverse transcribed to yield complementary DNA (cDNA). The undiluted cDNA solution was subsequently subjected to PCR amplification for IL-8 and GAPDH, using appropriate primers. The PCR products were analyzed by electrophoresis through 1.2% agarose gels containing 100 ng/mL ethidium bromide. The DNA bands corresponding to IL-8 and GAPDH were visualized using an ultraviolet transilluminator (Biorad) and their density was calculated using the Quantity One software (Biorad). IL-8 mRNA levels (as determined by RT-PCR) at the indicated time points are expressed as a ratio IL-8 band density versus GAPDH density.

As shown in FIG. 8, HT-29 cells treated with IL-1β alone showed an early and sustained increase in steady state IL-8 mRNA levels consistent with upregulation of IL-8 gene expression. This increase in IL-8 mRNA levels was inhibited by treatment with the S. boulardii supernatant.

S. Boulardii Culture Supernatant Prevents IκBα Degradation in IL-1β Stimulated THP-1 Cells.

The classical form of activated NF-κB is a heterodimer consisting of one p50 and one p65 subunit. In its inactive state NF-κB resides in the cytoplasm as a trimer bound to a member of the IκB family of inhibitor proteins. Cellular activation results in phosphorylation of IκB which is conjugated with ubiquitin and degraded by the proteasome. The active NF-κB dimer is then free to translocate to the nucleus, bind to DNA at κB sites and up-regulate gene transcription. We therefore examined whether S. boulardii supernatant could prevent IkB degradation following cellular activation.

THP-1 cells were seeded in 10 mm tissue culture dishes at a concentration of 8×10⁵ cells/mL and stimulated with IL-1β (10 ng/mL) or IL-1β plus filtered S. boulardii culture supernatant for the indicated time periods. Cytoplasmic extracts were then prepared and subjected to western blotting using an anti-IκBα antibody.

As shown in FIG. 9, the S. boulardii supernatant prevented IL-1β-induced IκBα degradation (5 to 30 minute time points). IκB degradation is a critical step towards NF-κB activation and nuclear translocation. Thus the ability of SAIF to prevent IκB degradation provides a mechanism for SAIF anti-inflammatory effects.

S. Boulardii Culture Supernatant Blocks NF-κB Activation in LPS Stimulated THP-1 Cells.

Since S. boulardii supernatant can prevent IκBα degradation following cell activation we next examined whether this inhibitory effect was associated with a reduction in NF-κB-regulated gene expression.

These experiments were performed in THP-1 monocytic cells since it has been reported that HT-29 colonocytes exhibit altered regulation of IκBα proteolysis. THP-1 cells (2×10⁷/mL) were transiently transfected with an NF-κB-responsive luciferase reporter gene construct using the DEAE-dextran procedure. Briefly, 2×10⁷ THP-1 cells were suspended in 1 mL prewarmed Tris-buffered saline and incubated for 10 minutes at 37° C. with 80 μg DEAE-dextran (Pharmacia). THP-1 cells were then transfected with 5 μg DNA of the luciferase NF-kB reporter plasmid. Transfection was stopped by adding 25 mL Tris-buffered saline. After washing, cells were cultured for 48 hours before stimulation. After stimulation with S. boulardii culture supernatant and/or purified LPS (100 ng/mL) for 5 hours, THP-1 cells (8×10⁶ cells per stimulus) were washed in PBS. Cell lysis and luciferase assay were performed using the Luciferase Assay System (Promega Corp.) according to the instructions ofthe manufacturer. Culture supernatants were also collected for IL-8 protein measurement by ELISA.

As is shown in FIG. 10, S. boulardii supernatant and filtered Sb supernatant both completely prevented LPS-induced NFκB-reporter gene activation in THP-1 cells (** denotes p<0.001 compared to control LPS stimulation alone, * denotes p<0.05 compared to LPS stimulation alone).

S. Boulardii Supernatant Reduces p65 Nuclear Translocation and NF-κB-DNA Binding.

Since S. boulardii supernatant prevents IκBα degradation, it is to be expected that NF-κB is retained in the cytoplasm and does not translocate to the nucleus to function. To test this hypothesis, we determined p65 protein levels in nuclear extracts of THP-1 cells stimulated for 4 h in the presence or absence of S. boulardii supernatant. As is shown in FIG. 17 (left panel), IL-1β stimulation results in rapid increase of p65 in the nucleus, starting at 5 minutes with a peak at 20 minutes. In contrast, in cells co-treated with S. boulardii supernatant the amount of p65 protein in the nucleus was less at all time points studied (FIG. 17, right panel). These findings, together with the IκKα degradation results (FIG. 9), indicate that the p65 NF-κB subunit is retained in the cytoplasm in S. boulardii supematant-treated THP-1 cells.

We next determined whether the observed reduction the amount of p65 in the nucleus results in attenuated NF-κB-DNA binding activity. After THP-1 cells were stimulated with IL-1β (10 ng/ml) for 1 hour, in the presence or absence of S. boulardii supernatant, NF-κB DNA binding activity in nuclear extracts was determined by EMSA. As shown in FIG. 18 (left panel), NF-κB-DNA binding is rapidly induced (5 min) following IL-1β stimulation; the activation peaks at 20 min and declines by 60 min. Co-treatment with S. boulardii supernatant results in marked reduction of NF-κB-DNA binding at all studied time points.

Purification and Characterization of S. Boulardii Anti-Inflammatory Factor (SAIF).

The Active Factor has Molecular Weight of <10 kD.

S. boulardii supernatant was produced as described above, the pH neutralized (to pH 7.0) with NaOH (35 mM), filtered through a 0.22 μm filter (Fisher Scientific, Agawam, Mass.), and then fractionated through a 10 kD filter (Millipore, Bedford, Mass.). Data shown are IL-8 protein levels (pg/mL) in HT-29 cell conditioned media.

Inhibitory activity was consistently retained in the <10 kD fraction (as shown in FIG. 11, * denotes P<0.001 compared to IL-1 alone by Student's t test). The finding that the inhibitory factor has a molecular mass of <10 kDa was further supported by dialysis of the supernatant against PBS, pH: 7.4, through a 12 kD dialysis membrane which resulted in loss of inhibitory activity.

The Active Factor is Heat Stable.

As shown in FIG. 12, the filtered S. boulardii supernatant did not lose its activity when heated to 100° C. (boiled) for 5 minutes. Data are shown as IL-8 protein levels (pg/mL) in HT-29 cell conditioned media (p<0.001 by ANOVA; * denotes p<0.001 compared to IL-1 alone (Bonferroni)). There is no significant difference in the inhibitory activity between filtered yeast supernatant and boiled filtered yeast supernatant (p>0.05).

The Lipid fraction of the S. Boulardii Supernatant is not Active.

Lipids were extracted from the <10 kD fraction of the filtered S. boulardii supernatant by liquid-liquid extraction using 6 volumes of chloroform-methanol (2:1, v/v) in a glass tube. After centrifugation at 800×g for 3 min, the resulting lower phase (organic phase) was aspirated and transferred to a separate tube. The organic solvents were then evaporated in the presence of N₂ and the dried material was reconstituted in HT-29 media by sonication. In some cases the organic phase was subjected to a second cycle of the same procedure (double lipid extraction).

As shown in FIG. 13, the <10 kD fraction of the S. boulardii supernatant is active (p<0.001 by ANOVA; * denotes p<0.001 compared to IL-1 alone by Bonferroni test). In contrast, lipids extracted from the <10 kD fraction do not show any inhibitory activity (p>0.05 compared to IL-1 alone). Data shown are IL-8 protein levels (pg/mL) in HT-29 cell conditioned media.

The Active Factor is a Glycosylated Compound.

Density gradient ultracentrifugation in cesium chloride (CsCl) has been used to separate highly glycosylated epithelial glycoproteins (mucins) from lipids and proteins/serum-type glycoproteins in respiratory secretions and gastrointestinal mucus. This method is based on the difference in buoyant density between proteins (˜1.3 g/ml) and carbohydrates (˜1.6 g/ml). Heavily glycosylated mucins containing ˜80% carbohydrate have a buoyant density of ˜1.5 g/ml. This method has the advantage that after the separation there is almost 100% recovery of material unlike in conventional chromatography methods.

Solid CsCl (42% w/w) was added to 8 mL of the <10 kD fraction of filtered S. boulardii supernatant and the solution (9 mL) was subjected to ultracentrifugation (Beckmann Ultracentrifuge) at 40,000 rpm for ˜68 hours. After centrifugation, fractions of 1 mL each were recovered by aspiration from the top and aliquots of the fractions weighed to determine density. An insoluble film (presumably lipid) was found sticking to the sides of the uppermost portion of the tube but this remained undisturbed during recovery of the fractions. Neutral sugar content of the fractions was determined by the phenol-sulfuric acid method as originally described by Dubois et al and recently miniaturized for use with microsample plate reader. Briefly, 25 μL of a 5% phenol solution was added to 25 μL of the fractions placed in the wells of a microtiter plate. After gentle mixing, the plate was placed on ice and 125 μl of concentrated sulfuric acid was added to each well. The plate was again stirred gently and placed in a 80° C. oven for 30 min after which the absorbance at 490 nm was determined using a plate reader. Standards of galactose solution containing 10-200 μg/mL were used and measurements were made in duplicate.

The fractions were dialyzed using the microcon3 device (molecular weight cut-off 3 kDa, Millipore, Bedford, Mass.) and tested for their inhibitory effect on IL-8 secretion by L-1-stimulated HT-29 monolayers (incubation time 12 h).

As shown in FIG. 14, all fractions (1 to 9) showed some inhibitory activity. However, greater inhibitory activity (almost complete blockage of IL-8 production) was observed with the last two fractions suggesting that the active factor is a dense, glycosylated compound, such as a glycan or a glycopeptide, and not a proteinaceous compound.

Further Evidence that the Active Factor is a Small Dense Glycosylated Compound.

Following cesium chloride gradient separation, the more dense fractions (7, 8 and 9; see FIG. 14) were pooled and further separated through a Biogel P-30 column. Hexose (neutral sugars, shown as μg/ml) and protein levels (shown as μg/ml) were measured in the resulting 18 fractions that were also tested for their ability to inhibit IL-8 protein production in IL-1β-stimulated HT-29 monolayers.

As shown in FIG. 15, the fractions that contained the highest levels of hexose (neutral sugars) and protein (#10, 11 & 12) were active in inhibiting IL-8 production (ANOVA, p<0.0001. Bonferroni tests for fractions 10, 11 and 12; p<0.01, p<0.001, and p<0.001 respectively compared to IL-1β stimulation alone).

These data provide further evidence that the active factor is a glycosylated compound, such as a glycan or a glycopeptide, and not a proteinaceous compound.

Fractionation Using a Biogel P-2 Column Indicates that the S. boulardii Anti-Inflammatory Factor has a Molecular Weight of <1 kDa.

Following cesium chloride gradient separation, the more dense fractions (7, 8 and 9, see FIG. 14) were pooled and eluted through a Biogel P-2 column (fractionation range 100-1800 Daltons) in order to achieve better separation than the Biogel P-30 column.

Hexose (neutral sugars, shown as μg/ml) and protein levels (shown as μg/ml) were measured in the resulting 15 fractions that were also tested for their ability to inhibit IL-8 protein production in IL-1β-stimulated HT-29 monolayers.

As shown in FIG. 16, fractions 10 and 11 potently inhibited IL-8 production (ANOVA, p=0.0003. Bonferroni test for fractions 10 & 11; p<0.05 for each compared to IL-1β stimulation alone; p>0.05 for all other fractions).

Under the same conditions, vitamin B12 (molecular weight 1,355 D) eluted from the P-2 column in a peak with maximum at fraction # 6. We conclude that the active factor which elutes in fractions 10, 11 and 12 has a molecular weight of less than 1,000 Da.

Enzyme Analysis of SAIF

We treated S. boulardii supernatant containing SAIF with proteinases, glycosidases, and other enzymes to identify which, if any, would result in the loss of SAIF-mediated inhibition of IL-8 production following stimulation of cells with IL-1β. Our results indicate that proteinases (e.g., proteinase K and chymotrypsin) do not eliminate SAIF activity.

Our results indicate that treatment of SAIF-containing S. boulardii supernatant with individual glycosidases (e.g., α-mannosidase, α-galactosidase, β-galactosidase, and β-N-acetylglucosaminidase) does not result in a loss of SAIF activity, while treatment of SAIF-containing S. boulardii supernatant with a mixture of glycosidases (e.g., α-mannosidase, β-mannosidase, α-glucosidase, β-glucosidase, α-galactosidase, β-galactosidase, α-L-fucosidase, β-xylosidase, α-N-acetylglucosaminidase, β-N-acetylglucosaminidase, α-N-acetylgalactosaminidase, and β-N-acetylgalactosaminidase) does result in a loss of SAIF activity, indicating that SAIF is a glycan or a glycopeptide, but not a polypeptide (see FIG. 20).

We have also tested a mixture of deglycosylases (e.g., PNGase F, O-glycosidase, sialidase, β-galactosidase, glucosaminidase, and endo F1) for their effect on the activity of SAIF. This mixture did not result in a loss of SAIF activity.

Finally, we also tested alkaline phosphatase, DNAse, aryl-sulfatase (contaminated with β-glucuronidase), 2-O-sulfatase, and β-glucuronidase for their effect on SAIF activity. Only treatment of SAIF-containing S. boulardii supernatant with aryl-sulfatase resulted in a loss of SAIF activity (see FIG. 21). This suggests that SAIF is a sulfated glycan.

Chemical Composition of the S. boulardii Anti-Inflammatory Factor

The small molecular size of the active factor is beyond the limits of resolution by conventional gel filtration and electrophoresis techniques. We therefore subjected the active fraction 11 from the Biogel P-2 column, to matrix assisted laser desorption time of flight (MALDI-TOF) mass spectroscopic examination to obtain a more accurate estimation of the MW. This technique disclosed a prominent peak with a mass of 774, supporting our conclusions from the gel filtration experiment that the active factor has a MW<1000 D.

We next determined the chemical composition of the active fractions 10 and 11 and the less active fraction 12 by subjecting them to amino-acid analysis using standard protocols, as well as carbohydrate analysis using gas chromatography-mass spectrometry (GC-MS). The results are shown in Tables 1 and 2.

TABLE 1
Amino acid composition of Biogel P-2 fractions (nanomole %) as
determined by the amino acid analyzer

| | Fraction #10 | | Fraction #11 | | Fraction #12 | |
|---|---|---|---|---|---|---|
| Asx | 15 | (15) | 8 | (4) | 4 | (3) |
| Glx | 5 | (5) | 5 | (6) | 8 | (7) |
| Ser | 16 | (18) | 6 | (4) | 3 | (3) |
| Gly | 8 | (8) | 6 | (5) | 9 | (12) |
| His | 5 | (5) | 4 | (2) | 4 | (nd) |
| Arg | 11 | (18) | 28 | (29) | 15 | (16) |
| Thr | 7 | (9) | 3 | (2) | 2 | (2) |
| Ala | 6 | (5) | 4 | (3) | 4 | (4) |
| Pro | 3 | (2) | 6 | (3) | 11 | (3) |
| Tyr | 1 | (nd) | 2 | (11) | 18? | (34) |
| Val | 2 | (2) | 1 | (3) | 1 | (2) |
| Met | 2 | (nd) | 1 | (2) | Nd | |
| Cys | 3 | (nd) | Nd | | Nd | |
| Ileu | 2 | (2) | 1 | (3) | 1 | (2) |
| Leu | 5 | (3) | 4 | (1) | 4 | (2) |
| Phe | 6 | (6) | 21 | (20) | 11 | (8) |
| Lys | 3 | (nd) | 2 | (nd) | 2 | |

nd = not detected

TABLE 2
Monosaccharide analysis by gas chromatography-mass spectroscopy
(GC-MS) of Biogel P2 fractions (nm/ml)

| | Fraction #10 | Fraction #11 | Fraction #12 |
|---|---|---|---|
| Arabinose | 20 | 19 | 9 |
| Ribose | 151 | 10 | 3 |
| Xylose | 50 | 4 | 2 |
| Mannose | 126 | 19 | 2 |
| Galactose | 888 | 139 | 18 |
| Glucose | 1111 | 142 | 20 |

As shown in Table 1 above, in fraction #11, the most active fraction, Arg and Phe are the predominant amino acids and are present in close to a 1:1 molar ratio. In fraction #12, these two amino acids are also present in close to a 1:1 molar ratio.

To verify our initials results, we obtained another set of active fractions, following the same procedures, and subjected them to amino-acid analysis using the same protocol. As shown in table 1, the analysis gave almost identical results (values in parentheses).

As shown in Table 2 above, galactose and glucose are the predominant neutral sugars and in all three fractions are present in close to a 1:1 molar ratio.

In conclusion, we have identified a compound, which we term SAIF, that is derived from yeast and has a molecular weight of <1 kD. Our data indicate that SAIF is a water soluble, stable glycan or glycopeptide that inhibits IκB degradation, prevents NF-κB activation, and attenuates pro-inflammatory signaling in host cells. Therefore, we conclude that SAIF is a useful pharmacologic agent for treating inflammatory diseases and disorders.

All publications and patent applications mentioned in this specification are herein incorporated by reference to the same extent as if each independent publication or patent application was specifically and individually indicated to be incorporated by reference.

1. An isolated soluble anti-inflammatory factor (SAIF) compound, wherein said SAIF compound is characterized as being a glycan or glycopeptide having a molecular weight from 500 to 1000 daltons, is present in the extract from a yeast cell, has SAIF biological activity in a cell contacted with a pro-inflammatory agent or in a cell of a patient having an inflammatory condition, and is heat stable.

2. The SAIF compound of claim 1, wherein said compound inhibits NF-κB activation.

3. The SAIF compound of claim 1, wherein said compound inhibits IκB degradation.

4. The SAIF compound of claim 1, wherein said pro-inflammatory agent is a factor produced by a bacterium, virus, or parasite.

5. The SAIF compound of claim 4, wherein said pro-inflammatory agent is selected from the group consisting of lipopolysaccharide (LPS), C. difficile Toxin A, and C. difficile Toxin B.

6. The SAIF compound of claim 1, wherein said pro-inflammatory agent is a cytokine selected from the group consisting of interleukin 1-β (IL-1β) and tumor necrosis factor -α (TNF-α).

7. The SAIF compound of claim 1, wherein said compound comprises a galactose moiety.

8. The SAIF compound of claim 1, wherein said compound comprises a glucose moiety.

9. The SAIF compound of claim 1, wherein said compound comprises a hexose moiety.

10. The SAIF compound of claim 1, wherein said compound comprises a neutral sugar.

11. The SAIF compound of claim 1, wherein said compound comprises a sulfate moiety.

12. The SAIF compound of claim 1, wherein said compound is purified from yeast.

13. The SAIF compound of claim 1, wherein said yeast cell is a member of the genus Saccharomyces.

14. The SAIF compound of claim 13, wherein said yeast cell is Saccharomyces boulardii.

15. The SAIF compound of claim 14, wherein said yeast cell is yeast cell has the biological characteristics of A.T.C.C. Deposit No. MYA-796 or A.T.C.C. Deposit No. MYA-797.

16. The SAIF compound of claim 1, wherein exposure of said compound to glycosidases attenuates the biological activity of said compound.

17. The SAIF compound of claim 1, wherein exposure of said compound to an arylsulfatase attenuates the biological activity of said compound.

18. The SAIF compound of claim 1, wherein exposure of said compound to a proteinase does not attenuate the biological activity of said compound.

19. The SAIF compound of claim 18, wherein said proteinase is proteinase K or chymotrypsin.

20. The SAIF compound of claim 1, wherein exposure of said compound to a deglycosylase does not attenuate the biological activity of said compound.

21. The SAIF compound of claim 20, wherein said deglycosylase is PNGase F, O-glycosidase, sialidase, β-galactosidase, glucosaminidase, or endo F1.

22. The SAIF compound of claim 1, wherein exposure of said compound to an alkaline phosphatase, a DNAse, a 2-O-sulfatase, or a β-glucuronidase does not attenuate the biological activity of said compound.

23. The SAIF compound of claim 1, wherein said compound is not a polypeptide.

24. The SAIF compound of claim 1, further comprising a pharmaceutically acceptable carrier or diluent.

25. A method of purifying a soluble anti-inflammatory factor (SAIF) compound, wherein said SAIF compound is characterized as being a glycan or glycopeptide having a molecular weight from 500 to 1000 daltons, is secreted from a yeast cell, has SAIF biological activity in a cell contacted with a pro-inflammatory agent or in a cell of a patient having an inflammatory condition, and is heat stable, said method comprising:
(a) providing a yeast culture;
(b) incubating said yeast culture in growth medium, wherein yeast in said yeast culture secrete said SAIF compound into said medium;
(c) removing said yeast from said medium to produce a supernatant comprising said SAIF compound; and
(d) isolating a fraction having SAIF biological activity.

26. The method of claim 25, wherein after step (d) said method further comprises precipitating said SAIF compound from said fraction.

27. The method of claim 25, wherein after step (d) said method further comprises using reverse phase chromatography to further purify said SAIF compound.

28. The method of claim 25, wherein in step (a) said yeast culture comprises cells of the genus Saccharomyces.

29. The method of claim 28, wherein said cells are Saccharomyces boulardii.

30. The method of claim 29, wherein said cells have the biological characteristics of A.T.C.C. Deposit No. MYA-796 or A.T.C.C. Deposit No. MYA-797.

31. The method of claim 25, wherein in step (b) said yeast culture is incubated in RPMI medium for 1 hour to 48 hours at a temperature in the range of 30° C. to 42° C.

32. The method of claim 31, wherein in step (b) said yeast culture is incubated for 24 hours at 37° C.

33. The method of claim 25, wherein in step (c) said removing comprises centrifugation of said yeast culture at 5,000 rpm to 10,000 rpm for 5 min. to 30 min.

34. The method of claim 33, wherein said centrifugation is at 7,400 rpm for 15 min.

35. The method of claim 25, wherein after step (c), said method further comprises filtering said supernatant through a 22 μm filter to produce a filtered supernatant.

36. A SAIF compound, said compound isolated by the method of claim 25.

37. The SAIF compound of claim 36, wherein said compound is substantially pure.

38. The SAIF compound of claim 36, further comprising a pharmaceutically acceptable carrier or diluent.

39. The SAIF compound of claim 37, further comprising a pharmaceutically acceptable carrier or diluent.

40. A method of treating or preventing an inflammatory condition in a subject in need thereof, said method comprising administering a SAIF compound to said subject, said SAIF compound administered at a dosage sufficient to elicit SAIF biological activity in said subject.

41. The method of claim 40, wherein said inflammatory condition is an inflammatory disease or disorder, or results from an injury.

42. The method of claim 41, wherein said inflammatory disease or disorder is inflammatory bowel disease.

43. The method of claim 42, wherein said inflammatory bowel disease is selected from the group consisting of Crohn's disease, ulcerative proctitis, ulcerative colitis, and microscopic colitis.

44. The method of claim 41, wherein said injury is a gastrointestinal injury.

45. The method of claim 44, wherein said gastrointestinal injury is caused by an infectious agent.

46. The method of claim 45, wherein said infectious agent comprises a bacterium, a virus, or a parasite.

47. The method of claim 44, wherein said gastrointestinal injury is caused by a toxin.

48. The method of claim 47, wherein said toxin is a Clostidium difficile toxin.

49. The method of claim 48, wherein said Clostridium difficile toxin is Toxin A or Toxin B.

50. The method of claim 41, wherein said inflammatory disease or disorder comprises the skin or musculoskeletal system.

51. The method of claim 50, wherein said inflammatory disease or disorder is selected from the group consisting of psoriasis, dermatitis, rheumatoid arthritis, and degenerative joint disease.

52. The method of claim 41, wherein said inflammatory disease or disorder is asthma.

53. A method of treating or preventing a patient undergoing chemotherapy, said method comprising administering to said patient a SAIF compound in combination with a chemotherapeutic agent.
